(19) United States
(12) Patent Application Publication
(10) Pub. No.: US 2008/0015699 A1
(43) Pub. Date: Jan. 17, 2008

(54) Postero-Lateral Intervertebral Disc Prosthesis

(76) Inventor: Gilles Voydeville, Nancy (FR)

Correspondence Address: HESLIN ROTHENBERG FARLEY & MESITI PC, 5 COLUMBIA CIRCLE, ALBANY, NY, 12203, US

(21) Appl. No.: 10/580,919

(22) PCT Filed: Nov. 26, 2004
(86) PCT No.: PCT/FR04/50624

§ 371 Date: Mar. 2, 2007

(30) Foreign Application Priority Data
Nov. 28, 2003 (FR) 0314210

(51) Int. Cl. A61F 2/44; A61B 17/58

(52) U.S. Cl. 623/17.16; 606/99

(57) ABSTRACT

The invention relates to a postero-lateral intervertebral disc prosthesis characterised in that: the insert (1) has a fixed planar section on the lower vertebral plateau, the element (3) has a lower planar surface for support on the insert (1) with a limited capacity for translational displacement and an upper surface with a generally hemispherical form, the insert (2) has a fixed planar section on the upper vertebral plateau and opposite thereto, a concave surface for cooperating with the hemispherical surface of the element (3), with the possibility of multi-directional articulation. The inserts (1) and (2) and the insert (3) are in the form of a disc with a diameter of less than about 30 mm and, when juxtaposed, define a total height of about 11 to 15 mm to permit introduction by an initially postero-lateral route.

The invention relates to the technical field of intervertebral implants.

Disc degeneration may appear in the form of different disorders, among which one can note lumbago or sprain with disc tearing, disc hernia and disc insufficiency.

The first stage is a matter for medicine, the second for medicine or exeresis surgery and the third is a matter for conservative treatments that often lead to failures, or for surgical treatments which are mainly arthrodesis or prosthesis.

The operative indication is based on clinical signs of long-term lumbalgia associated more or less with radiculalgia, radiographic signs of disc pinching or osteophytoses, scannographic signs of disc degeneration with a disc cavity, and signs in nuclear magnetic resonance of disc degeneration with modifications of the vertebral plateaux that become inflammatory.

Indication greater than 18 years, less than 55 years, Modic I stage, discography painful upon injection, scanner showing conservation of the joints.

Treatment by arthrodesis produces good results but its effect is to eliminate a degree of mobility of the rachis. It has the advantage of good positioning of the rachis and of being painless. On the other hand, given that it constitutes merely a blockage of the joint, it may cause the adjacent joints to suffer.

If one considers coxarthrosis in parallel, while it benefited initially from arthrodeses that produced results in terms of pain, it was relegated to obsolescence as soon as the articulated hip prosthesis was developed.

In order to overcome these drawbacks generated by arthrodesis, different types of disc prostheses have been proposed for a number of years. These prostheses eliminate pain and restore mobility.

Various technical solutions have been proposed.

U.S. Pat. No. 6,368,350 discloses a prosthesis the external configuration of which may have any shape and any size, and which is to be fitted by an anterior route, with a convex fixed component and a flat concave intermediate component. A disc prosthesis is obtained whose centre of rotation is not located at the appropriate place on the lower vertebral plateau, as has been shown by the work of Professor Rene LOUIS (Springer Verlag).

The PCT document WO 01/011893 discloses a rectangular-shaped prosthesis which is fitted by an abdominal anterior route and which constitutes a fixed intermediate prosthesis.

The patent FR 2824261 concerns a prosthesis whose shape is pseudo-rectangular, with a cylindrical-shaped intermediate piece having a convex end at its upper end and a concave end at its lower end.

Furthermore it is apparent from various publications that:

the work of Professor Rene LOUIS (Springer Verlag) has demonstrated that the centre of rotation of a disc is located on the lower vertebral plateau;

the work of PEARCY (Spine 1988) has shown that this centre of rotation on the lower vertebral plateau tends to be located in the posterior third of the vertebra;

the work of the applicant of the present application, as published in the EJOST 2000, 10:167-176, analysing the normal functioning of the rachis, demonstrates the coupled translational and rotational movements in flection-extension and lateral inflection movements.

Starting out from this prior art, one of the problems that the invention sets out to solve is, first, to make a disc prosthesis that is small enough in size and that functions in a manner close to physiology; and, second, to implant this prosthesis by a new posterior approach route.

According to the invention, the disc prosthesis is round and has a diameter which is less than 30 mm in order to be able to be inserted by a posterior route, given that the nerve elements—dural sac, roots, and also vessels—do not make it possible to fit a larger prosthesis at this location.

It comprises three sections:

a fixed planar section on the lower vertebral plateau;

an intermediate section whose lower surface is planar and whose upper surface is totally hemispherical, capable of moving on the lower vertebral plateau, and limited by a central pin;

a fixed section on the upper vertebral plateau, the joint of which with the sphere is made by a concave surface;

the height of this prosthesis being limited to 11 to 15 mm so as to be able to be introduced by the posterior route.

A facet osteotomy which, if it did not exist, would not allow the prosthesis to be fitted by a posterior route. This facet osteotomy allows the prosthesis to be passed through and provision is made for reconstruction of the facets so as to totally restore the anatomy.

This facet osteotomy comprises an osteotomy of the lower facet of the upper vertebra and a lower osteotomy of the upper facet of the lower vertebra.

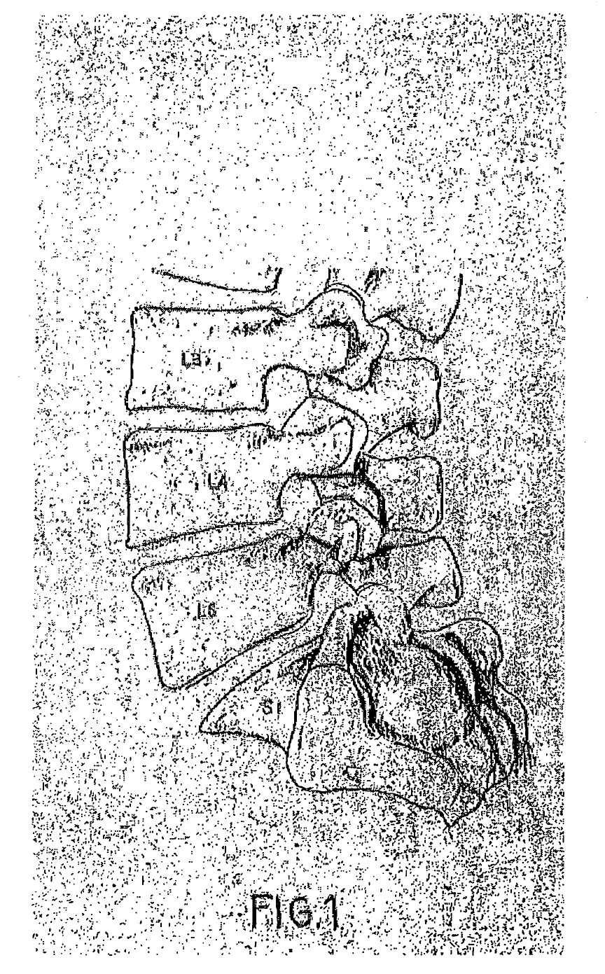

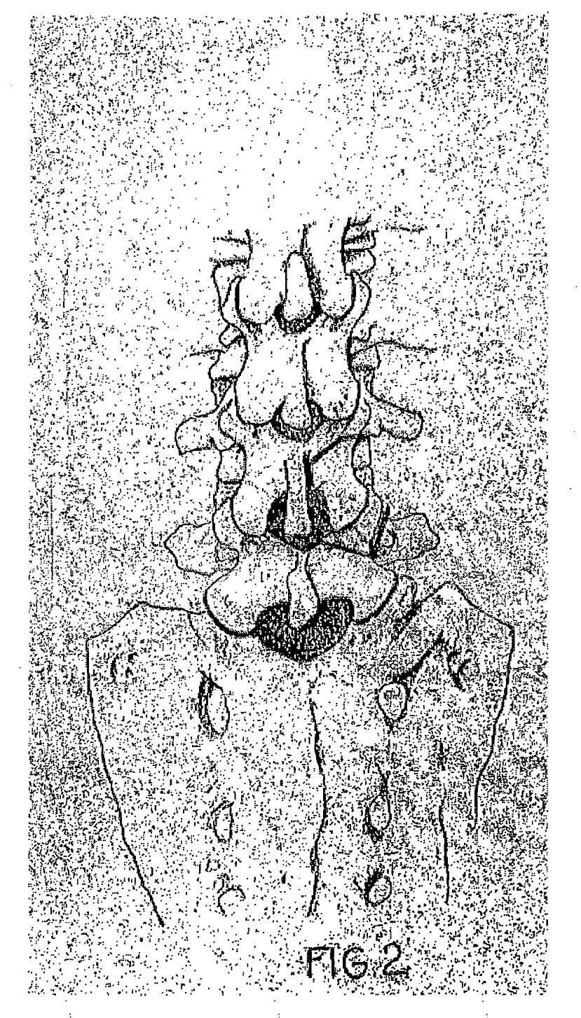

It is a biplanar osteotomy which incises from inside on the blade, incising the latter over 5 mm. Then this osteotomy becomes horizontal and will cut the blade, which at that point in time is protected on its lower face by a root-type or spatula-type retractor, and will cut the round part of the insertion from the joint segment on the pedicle, once the underlying root has been protected by a right-angled, blunt-edged Homman retractor (FIGS. 1 and 2).

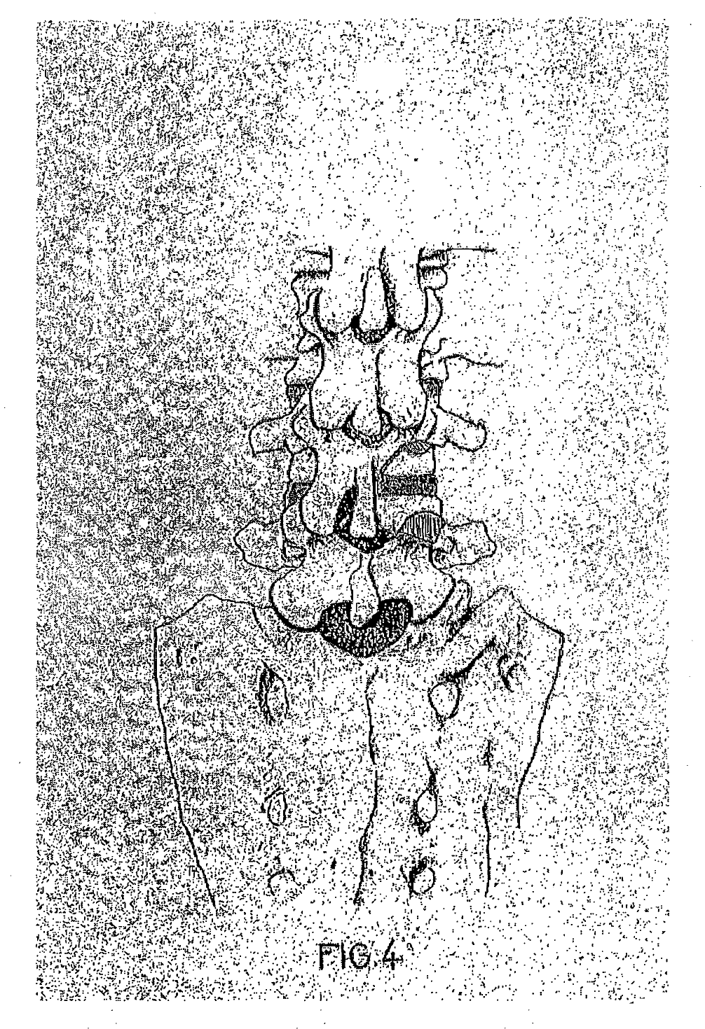

The fragment thus prepared in shown in FIGS. 6 and 7.

At this point it is prepared on the table and perforated with a hole so as to be able to be re-inserted in a second stage.

Once the hole has been prepared, this fragment is re-positioned and, by means of a small stylet, the orifice in the insertion of the pedicle is prepared with a hammer so as to obtain an insertion of 7 to 8 m.

The second stage of the osteotomy concerns the osteotomy of the lower articular facet which, in the present case, is the upper articular facet of vertebra L5.

It is likewise a biplanar osteotomy the base of which has a slightly oblique antero-posterior orientation beneath the articular capsule; this osteotomy is performed until the perpendicular of the transverse of vertebra L5.

The content of the spinal canal is secured by a retractor, a blade or a spatula, and the curved Cauchois osteotome, which is 12 mm thin, is inserted on the lateral face of the articular facet, inclined forwardly and obliquely at 30° in relation to the vertical; at this time the chisel is struck inwardly so as to separate the articular facet (Description of the orientation in FIGS. 1 and 2 for the front and profile).

The fragment of joint that is obtained is described in FIGS. 5a and 5b.

The pre-perforation is done on the preparation table by means of a 2.7 mm drill bit.

The osteotomy thus performed releases the dural sac on the inside, outside of the root, which in this particular case is the root L4.

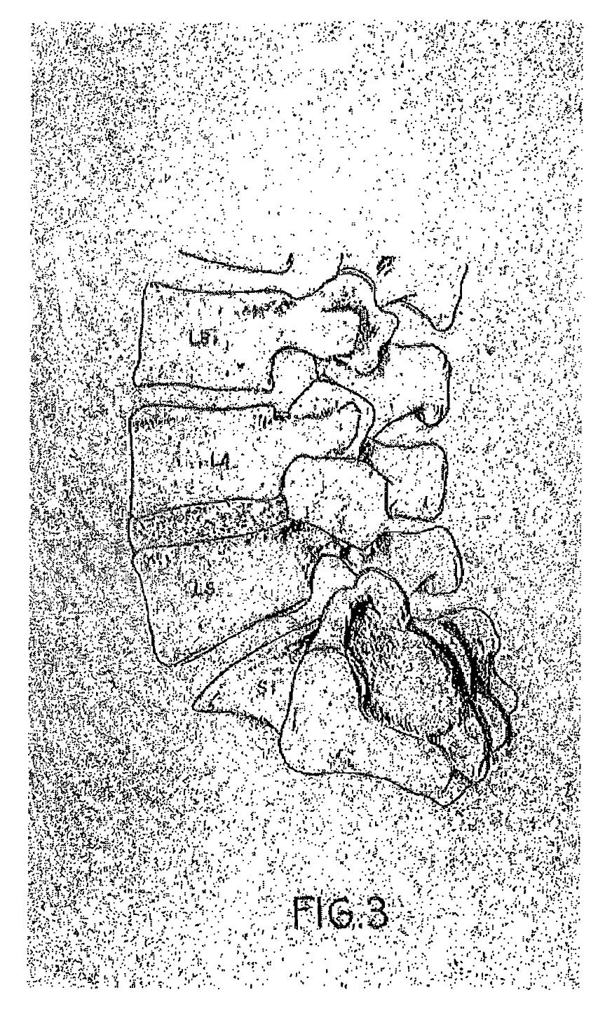

A view of the appearance obtained is given in the sketches in FIGS. 3 and 4.

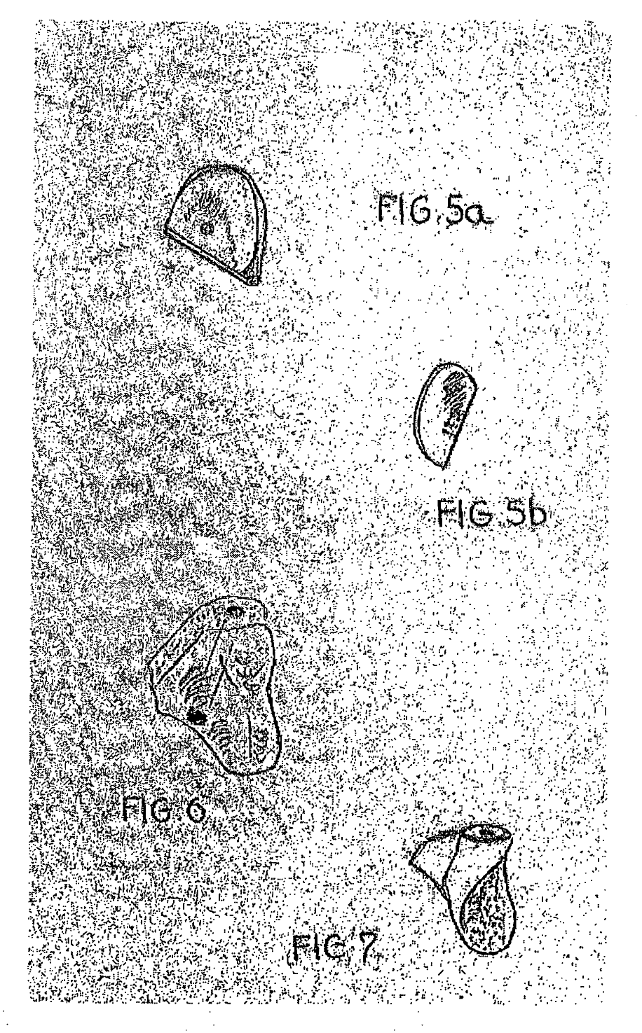

The retractor especially developed in FIG. 8 will make it possible to fix an orthostatic retractor on the conserved over-lying blade, with the internal section of the orthostatic retractor separating the dural sac and the external part separating the root, in this particular case L4.

Thus a discal space 25 to 30 mm wide is created over the whole height of the disc, as shown in FIG. 9, which will therefore be the postero-lateral approach route following facet osteotomy.

It is therefore apparent that the problem which the invention sets out to solve is to devise an articular prosthesis that can be fitted by a postero-lateral approach route, the objective being to respect physiology by allowing antero-posterior translation during flection and medio-lateral translation during lateral inflection.

In order to solve the problem of ensuring orientation and self-centering, the element mounted with an orientation and self-centering capability is formed by a core of a generally hemispherical shape capable of co-operating with a recess having a shape that complements one of the inserts, said core having fittings for stable positioning with the other insert, said inserts having a generally circular shape in the form of a disc.

Starting out from this basic concept, the positioning fittings of the core are constituted by coupling means which cooperate with complementary means of the insert to provide either a fixed connection or a mobile connection.

It is advantageous for the coupling means capable of providing a fixed connection to be constituted by complementary clipping means.

It is advantageous for the coupling means capable of providing a mobile connection to be constituted by recesses and projections that act as a pivot pin, having a capacity for translational displacement.

In order, to solve the problem of fitting the prosthetic implant by the posterior route, each of the inserts has, over its thickness, fittings to engage gripping and handling means, whereby the core also has, over its thickness, fittings to engage gripping and handling means.

The invention is described hereinafter in more detail by means of the figures of the attached drawings in which:

FIG. 1 is a partial view of a spinal column showing the articular osteotomies;

FIG. 2 is a front view corresponding to FIG. 1;

FIG. 3 is more particularly a side view of the disc after osteotomy of the facets;

FIG. 4 is a rear view according to FIG. 3 showing the disc after osteotomy of the facets;

FIGS. 5a and 5b are views of the exterior articular facet with screws;

FIG. 6 is a front view of the upper articular facet;

FIG. 7 is a profile view of the upper articular facet;

FIG. 8 shows an embodiment of the blade retractor for fixing an orthostatic retractor;

FIG. 9 shows the discal space obtained over the whole height of the disc which constitutes the postero-lateral approach route following osteotomy of the facets;

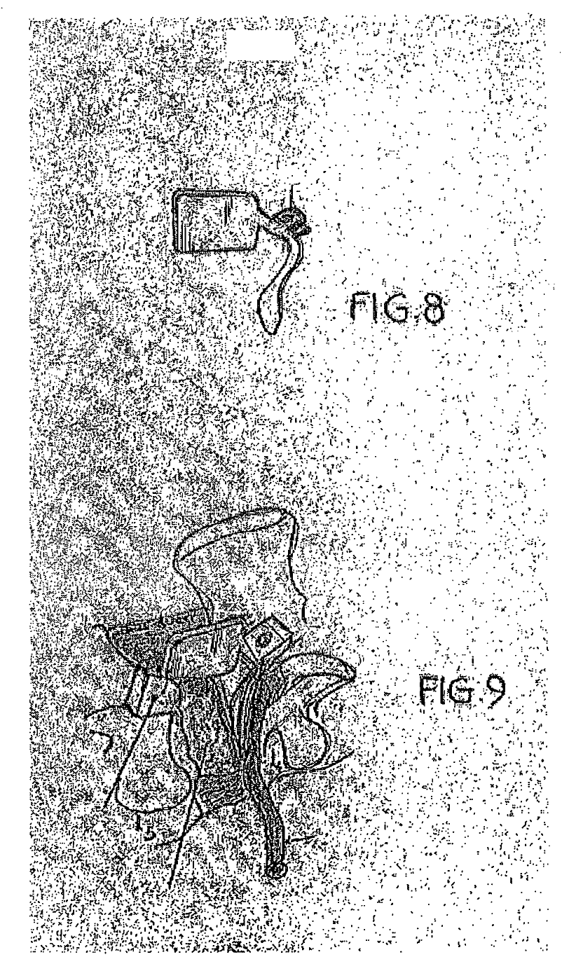

FIG. 10 shows an embodiment of the prosthesis according to the invention, fitted between two vertebral plateaux;

FIG. 11 is an exploded perspective view of the elements of the prosthesis in an embodiment;

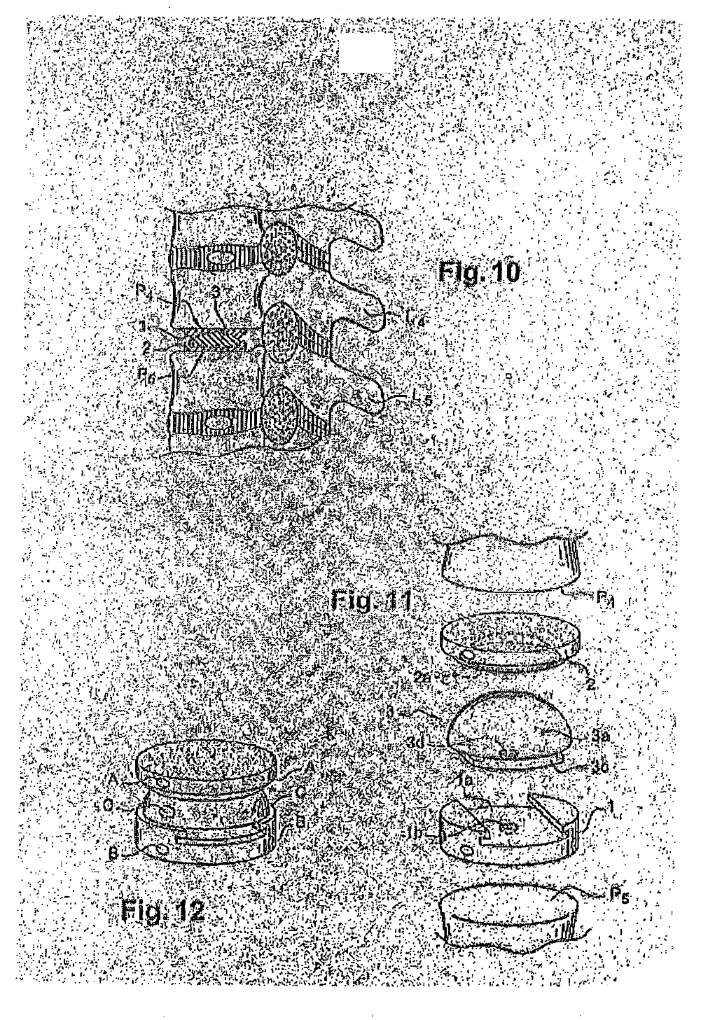

FIG. 12 is a perspective view corresponding to FIG. 2, following assembly of the elements;

FIG. 13 is a longitudinal cross-sectional view of another embodiment of the prosthesis, fitted between two vertebral plateaux;

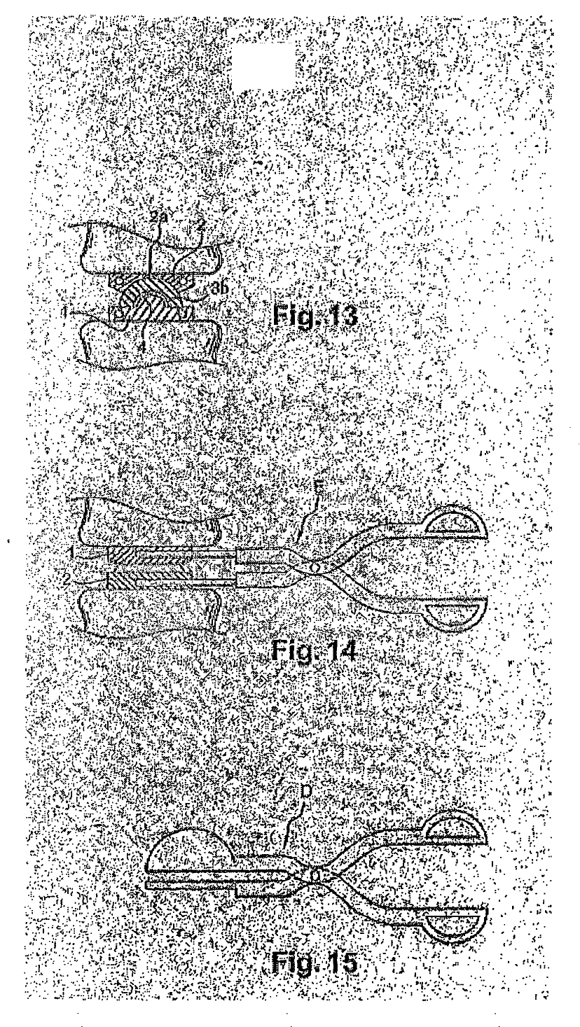

FIG. 14 is a schematic view showing the fitting of the inserts;

FIG. 15 is a schematic view showing the fitting of the articulation core or head.

The disc prosthesis according to the invention is shaped to be mounted between the vertebral plateaux (P4) and (P5) of any two vertebrae, for example L4 and L5. The postero-lateral prosthesis comprises two fixed inserts (1) and (2) forming one piece with the lower plateau (P4) and the upper plateau (P5), and subjected to an intermediate element (3) with an orientation and self-centering capacity. The whole of the prosthesis has a generally round shape, in respect of both the inserts (1) and (2) and the intermediate pivoting element (3). Thus inserts (1) and (2) take the form of discs made in several thicknesses and several diameters. The intermediate element (3) is formed by a sphere portion and has, for example, a generally hemispherical form. This element (3) can be made of high-density polyethylene, but without excluding other materials such as ceramic, diamond, chromium-cobalt alloy, . . . The same applies to the inserts (1) and (2). The hemispherical head (3) has at its base fittings for positioning with the insert (1), which are shaped to provide a mobile or fixed connection.

The hemispherical head (3), which acts as a core, has a hemispherical internal cavity (3b) on the side of its support face (3a) with insert (2) for cooperating with a complementarily-shaped pivot pin (4) which projects from the upper face of the insert (1). In this embodiment the core (3) is rotationally mobile. Similarly, the pivot pin (4) is dimensioned smaller than the hemispherical internal cavity in order to permit a translational movement of the head (3).

In the context of a fixed connection of the core (3), the latter may have, in combination with the insert (1), complementary coupling means. For example, as is shown by FIGS. 2 and 3 of the drawings, the insert (1) has a dovetail groove (1a) for free engagement, by sliding, of complementary means (3c) featured by the lateral edges of the base of the core (3). According to one embodiment, the core (3) may have a projecting stud (3d) capable of cooperating with a complementary recess (1b) in the groove (1a) in order to provide translational blocking of the core (3). Other means may evidently be provided.

However the core (3) is mounted in relation to the insert (1), that is, whether it is capable of mobility or not, the convex hemispherical cap (3a) of the core (3) cooperates with a complementarily-shaped concave cavity (2a) on the upper insert (2). The support faces of the inserts (1) and (2) evidently feature any type of fitting attaching to the corresponding support sections of the previously prepared vertebral plateaux. For example, the support face of the inserts (1) and (2) can exhibit a plurality of lugs covered with, for example, hydroxyapatite.

The intermediate connecting core (3) of the prosthesis may operate in antero-posterior flection, in lateral inflection and in axial rotation. This relative mobility allows the translational phenomena inherent in all movements of the rachis to be respected, as is apparent from a study published in the European Journal of Orthopaedic Surgery and Traumatology published in 2000.

This study made it possible notably to observe coupled movements, in particular during flection-extension, a translation of about 4 mm, which is completely respected by the prosthesis according to the invention, which has a corresponding antero-posterior translation. The lateral-inflection movements also require a lateral displacement of about 4 mm and, in axial rotation, a lateral displacement which varies from about 1.5 to 2 mm, according to the antero-postero and lateral axis, which is also respected by the characteristics of this prosthesis.

By way of information with no limiting effect, it is possible to make prostheses having diameters of 22 to 30 mm, with the average height of all the prostheses varying from 11 to 15 mm.

A basic insert (1) may have a thickness of about 3 mm in the case of a mobile prosthesis, and of about 3 to 6 mm on fixed prostheses. The upper insert (2) has a minimum thickness of approximately 1 mm at its centre and of up to 6 mm on the periphery.

The inserts (1) and (2) and the mobile core (3) advantageously feature fittings (A), (B) and (C) for engaging holding and handling means, for the fitting of the entire prosthesis, as indicated below.

The fitting of the prosthesis is done as follows:

Once the approach route has been completed, the disc is cut out over its entire height and over a width that may vary from approximately 22 to 28 mm, depending on the width of the disc and the available space. Since the dural sac and the root are retracted and the hemostasis of the branches of the lombar artery contiguous to the root has been effected, the operator places a right-angled retractor on the lateral edge of the disc in order to complete exposure. The opening of the disc is preceded by a hemostasis of the veinosi-vertebrales intemi plexus and of the lower foraminis intervertebralis veins.

The root L4, for example, is delicately retracted by any known and appropriate means in order not to injure the branches that extend from the root L4 to the lower root L5. Since the disc is open, the annulus is excised. The disc is then cureted by this postero-lateral route. Curetage is then performed at the front, at the centre and on the contralateral side.

The vertebral plateaux are then prepared, for example, with a cage-type knife, with a retractor that can be introduced either between the transverses or at the level of the disk in order to facilitate the exposure of the latter and freshening.

The operator then positions a first phantom disc prosthesis. Its positioning must be effected at the centre in the sagittal plane and in the antero-posterior direction, positioned slightly to the rear of the centre of the vertebral plateau, according to the work of Pearcy (“Movements of a lombo-spine geode by three dimensional X-way analysis” J. Biomed Eng 4: 107-112), or slightly farther forwards, right in the centre of the vertebral plateau if one considers the work of Rene Louis (Springer Verlag, Chirurgie du Rachis 1982) who both study the functioning of the normal disc.

The phantom is marked with brilliance amplifier, for example. If it is not completely marked, at that point in time markers are placed on the lower vertebral plateau to mark the external location and in the antero-posterior direction behind the posterior common vertebral ligament, which has not been incised in its median part, and contralateral at about 5 mm from the edge of the vertebra. The prosthesis, without the mobile core (3), is then put in place by means of a retractor of the type used in the field of cervical column surgery. After the prosthetic elements have been introduced with the retractor (E), a severance is effected in order to anchor the lugs of the inserts (1) and (2) in the vertebral plateaux (FIG. 5).

The positioning pincers are then replaced by severing pincers (D), the shape of which corresponds approximately to that of the mobile core and which are introduced after retraction of the two vertebral plateaux by an intervertebral retractor to push the inserts (1) and (2) against the corresponding parts of the vertebral plateaux.

The mobile core (3) is then introduced, either by sliding in the case of a fixed prosthesis, or by severance in the case of a mobile prosthesis, and is slid between the two inserts (1) and (2) that have been pre-positioned between the vertebral plateaux, as previously stated.

After having put in place the prosthesis, the movements are performed by traction on the spinous processes in order to visualize the functioning of said prosthesis in flection-extension, lateral inflection and possibly axial rotation. If the flap of the posterior common vertebral ligament has been conserved, it is folded back to protect the prosthesis from the dura mater.

Then the lower joint fragment which has been pre-perforated, and into which a stylet has been introduced to prepare the holding orifice in the pedicle, is put in place with a screw the length of which must be approximately 20 mm, with a flat head or double thread as in Scarff-type screws. Then the upper joint fragment, on the same principle, is screwed down by using the round part of the attachment of the joint to the pedicle, with a pre-perforation that had been performed initially and by a screw the length of which varies from 20 to 30, a flat-headed or rather double-threaded screw of the Scarff type.

The advantages are clearly apparent from the description, in particular we emphasize and remind the reader of:

the possibility of introducing the prosthesis by a posterior route, taking into account the minimum dimensions and its generally circular shape; it is consequently placed by a postero-lateral route and is perfectly centred by fluoroscopic marking;

compliance with physiology, given that the prosthesis has a centre of rotation that permits flection and antero-posterior and medio-lateral translation;

the possibility of posterior opening of the lumbar canal to fit the prosthesis, permitting curetage of the associated compression elements, which can be cureted only by this posterior route: narrow canal, disc hernia, osteophytes.

the use of the posterior route allows the plexus elements to be respected in order not to dissect the large vessels or the ureter, which may be injured by the anterior approach routes used in the prior art;

this prosthesis allows one to conserve the articular processes by osteotomizing them and re-fixing them according to a specific protocol.

1. Postero-lateral intervertebral disc prosthesis comprising an element (3) mounted with an orientation and self-centering capability between two inserts (1) and (2) disposed between the vertebral plateaux of two successive vertebral bodies, wherein:
the insert (1) has a planar section fixed on the lower vertebral plateau;
the element (3) has a lower planar surface for support, with a limited capacity for translational displacement on the insert (1), and an upper surface with a generally hemispherical form;
the insert (2) has a planar section fixed on the upper vertebral plateau and opposite thereto, a concave surface for cooperating with the hemispherical surface of the element (3), with the possibility of multi-directional articulation;
the inserts (1) and (2) and the insert (3) are in the form of a disc with a diameter of less than about 30 mm and, when juxtaposed, define a total height of about 11 to 15 mm to permit introduction by a postero-lateral approach route

2. Prosthesis according to claim 1, wherein the element (3) constitutes a core of a generally hemispherical shape capable of co-operating with the recess (2a) having a concave shape that complements the insert (2), said core (3) having a positioning stud (3d) that cooperates with a recess (1b) of the other insert (1) in order to permit axial rotation.

3. Prosthesis according to claim 1, wherein the positioning fittings of the core (3) are constituted by coupling means (3c) which cooperate with complementary means (1a) of the insert (1) to provide a fixed connection.

4. Prosthesis according to claim 1, wherein the positioning fittings of the core (3) are constituted by coupling means which cooperate with complementary means of the insert to provide a mobile connection, with a capacity for limited translational displacement

5. Prosthesis according to claim 3, wherein the coupling means capable of providing a fixed connection are constituted by complementary clipping means (3c)-(1a).

6. Prosthesis according to claim 4, wherein the coupling means capable of providing a mobile connection are constituted by recesses and projections that act as a pivot pin (2b)-(4).

7. Prosthesis according to claim 1, wherein each of the inserts (1) and (2) has, over its thickness, fittings (E1)-(E2) to engage gripping and handling means.

8. Prosthesis according to claim 1, wherein the core (3) has, over its thickness, fittings (E3) to engage gripping and handling means

9. Autostatic retractor for fitting a prosthesis, according to any one of claims 1 to 8, wherein it is shaped so as not to injure the neurological elements.
